SEQUENCE LISTING (8 sequences)

SEQ ID NO: 1 - DNA, 2817 bp, Corynebacterium glutamicum ATCC14752
  CDS (398)..(1864)
aatgaaataatcccctcaccaactggcgacattcaaacaccgtttcatttccaaacatcgagccaagggaaaagaaagcccctaagccccgtgttattaaatggagactctttggagacctcaagccaaaaaggggcattttcattaagaaaatacccctttgacctggtgttattgagctggagaagagacttgaactctcaacctacgcattacaagtgcgttgcgctgccaattgcgccactccagcaccgcagatgctgatgatcaacaactacgaatacgtatcttagcgtatgtgtacatcacaatggaattcggggctagagtatctggtgaaccgtgcataaacgacctgtgattggactctttttccttgcaaaatgttttccagcggatgttgagttttgcgacccttcgtggccgcatttcaacagttgacgctgcaaaagccgcacctccgccatcgccactagccccgattgatctcactgaccatagtcaagtggccggtgtgatgaatttggctgcgagaattggcgatattttgctttcttcaggtacgtcaaacagtgataccaaggtgcaagttcgagcggtgacctctgcgtatggcctgtactatacgcatgtggatatcacgttgaatacgatcaccatcttcaccaacatcggtgtggagaggaagatgccggtcaacgtgtttcatgttgtgggcaagttggacaccaacttctccaaactgtctgaggttgaccgtttgatccgttccattcaggctggtgctaccccgcctgaggttgccgagaaaattctggacgagttggagcaatcgcctgcgtcttatggtttccctgttgcgttgcttggctgggcaatgatgggtggcgctgttgctgtgctgttgggtggtggatggcaggtttccctaattgcttttattaccgcgttcacgatcattgccacgacgtcatttttgggaaagaagggtttgcctactttcttccaaaatgttgttggtggttttattgccacgctgcctgcatcgattgcttattctttggcgttgcaatttggtcttgagatcaaaccgagccagatcatcgcatctggaattgttgtgctgttggcaggtttgacacttgtgcaatctctgcaggacggcatcacgggcgctccggtgacagcaagtgcacgattttttgaaacactcctgtttaccggcggcattgttgctggcgtgggtttgggcattcagctttctgaaatcttgcatgtcatgttgcctgccatggagtccgctgcagcacctaattattcgtctacattcgcccgcattatcgctggtggcgtcaccgcagcggccttcgcagtgggttgttacgcggagtggtcctcggtgattattgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccttgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcaaagattcggtaataaaaggtaaaaatcaacctgcttaggcgtctttcgcttaaatagcgtagaatatcgggtcgatcgcttttaaacactcaggaggatccttgccggccaaaatcacggacactcgtcccaccccagaatcccttcacgctgttgaagaggaaaccgcagccggtgcccgcaggattgttgccacctattctaaggacttcttcgacggcgtcactttgatgtgcatgctcggcgttgaacctcagggcctgcgttacaccaaggtcgcttctgaacacgaggaagctcagccaaagaaggctacaaagcggactcgtaaggcaccagctaagaaggctgctgctaagaaaacgaccaagaagaccactaagaaaactactaaaaagaccaccgcaaagaagaccacaaagaagtcttaagccggatcttatatggatgattccaatagctttgtagttgttgctaaccgtctgccagtggatatgactgtccacccagatggtagctatagcatctcccccagccccggtggccttgtcacggggctttcccccgttctggaacaacatcgtggatgttgggtcggatggcctggaactgtagatgttgcacccgaaccatttcgaacagatacgggtgttttgctgcaccctgttgtcctcactgcaagtgactatgaaggcttctacgagggcttttcaaacgcaacgctgtggcctcttttccacgatttgattgttactccggtgtacaacaccgattggtggcatgcgtttcgggaagtaaacctcaagttcgctgaagccgtgagccaagtggcggcacacggtgccactgtgtgggtgcaggactatcagctgttgctggttcctggcattttgcgccagatgcgccctgatttgaagatcggtttcttcctccacattcccttcccttcccctgatctgttccgtcagctgc

SEQ ID NO: 2 - protein, 489 aa, Corynebacterium glutamicum ATCC14752
MLSFATLRGRISTVDAAKAAPPPSPLAPIDLTDHSQVAGVMNLAARIGDILLSSGTSNSDTKVQVRAVTSAYGLYYTHVDITLNTITIFTNIGVERKMPVNVFHVVGKLDTNFSKLSEVDRLIRSIQAGATPPEVAEKILDELEQSPASYGFPVALLGWAMMGGAVAVLLGGGWQVSLIAFITAFTIIATTSFLGKKGLPTFFQNVVGGFIATLPASIAYSLALQFGLEIKPSQIIASGIVVLLAGLTLVQSLQDGITGAPVTASARFFETLLFTGGIVAGVGLGIQLSEILHVMLPAMESAAAPNYSSTFARIIAGGVTAAAFAVGCYAEWSSVIIAGLTALMGSAFYYLFVVYLGPVSAAAIAATAVGFTGGLLARRFLIPPLIVAIAGITPMLPGLAIYRGMYATLNDQTLMGFTNIAVALATASSLAAGVVLGEWIARRLRRPPRFNPYRAFTKANEFSFQEEAEQNQRRQRKRPKTNQRFGNKR

SEQ ID NO: 3 - DNA, 1909 bp, Corynebacterium glutamicum ATCC13032
  CDS (280)..(1746)
agcttgcatgcctgcaggtcgactctagaggatcccccccctttgacctggtgttattgagctggagaagagacttgaactctcaacctacgcattacaagtgcgttgcgctgccaattgcgccactccagcaccgcagatgctgatgatcaacaactacgaatacgtatcttagcgtatgtgtacatcacaatggaattcggggctagagtatctggtgaaccgtgcataaacgacctgtgattggactctttttccttgcaaaatgttttccagcggatgttgagttttgcgacccttcgtggccgcatttcaacagttgacgctgcaaaagccgcacctccgccatcgccactagccccgattgatctcactgaccatagtcaagtggccggtgtgatgaatttggctgcgagaattggcgatattttgctttcttcaggtacgtcaaatagtgacaccaaggtacaagttcgagcagtgacctctgcgtacggtttgtactacacgcacgtggatatcacgttgaatacgatcaccatcttcaccaacatcggtgtggagaggaagatgccggtcaacgtgtttcatgttgtaggcaagttggacaccaacttctccaaactgtctgaggttgaccgtttgatccgttccattcaggctggtgcgaccccgcctgaggttgccgagaaaatcctggacgagttggagcaatcccctgcgtcttatggtttccctgttgcgttgcttggctgggcaatgatgggtggtgctgttgctgtgctgttgggtggtggatggcaggtttccctaattgcttttattaccgcgttcacgatcattgccacgacgtcatttttgggaaagaagggtttgcctactttcttccaaaatgttgttggtggttttattgccacgctgcctgcatcgattgcttattctttggcgttgcaatttggtcttgagatcaaaccgagccagatcatcgcatctggaattgttgtgctgttggcaggtttgacactcgtgcaatctctgcaggacggcatcacgggcgctccggtgacagcaagtgcacgatttttcgaaacactcctgtttaccggcggcattgttgctggcgtgggtttgggcattcagctttctgaaatcttgcatgtcatgttgcctgccatggagtccgctgcagcacctaattattcgtctacattcgcccgcattatcgctggtggcgtcaccgcagcggccttcgcagtgggttgttacgcggagtggtcctcggtgattattgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccctgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcagagattcggtaataaaaggtaaaaatcaacctgcttaggcgtctttcgcttaaatagcgtagaatatcgggtcgatcgcttttaaacactcaggaggatccttgccggccaaaatcacggacactcgtcccaccccagaatcccttcacgctgttgaagaggaaaccgcagccggggtaccg

SEQ ID NO: 4 - protein, 489 aa, Corynebacterium glutamicum ATCC13032; identical to SEQ ID NO: 2

SEQ ID NO: 5 - DNA, 21 bp, PCR primer
cccctttgacctggtgttatt

SEQ ID NO: 6 - DNA, 18 bp, PCR primer
cggctgcggtttcctctt

SEQ ID NO: 7 - DNA, 17 bp, PCR primer
gtaaaacgacggccagt

SEQ ID NO: 8 - DNA, 18 bp, PCR primer
ggaaacagctatgaccat

(19) United States
(12) Patent Application Publication
(10) Pub. No.: US 2002/0168731 A1
(43) Pub. Date: Nov. 14, 2002

(54) New nucleotide sequences coding for the thrE gene and process for the enzymatic production of L-threonine using coryneform bacteria

(75) Inventors: Petra Ziegler, Aachen (DE); Lothar Eggeling, Julich (DE); Hermann Sahm, Julich (DE); Georg Thierbach, Bielefeld (DE)

Correspondence Address: PILLSBURY WINTHROP LLP, 1600 TYSONS BOULEVARD, MCLEAN, VA, 22102, US

(73) Assignee: Degussa-Huls AG

(21) Appl. No.: 09/783,388

(22) Filed: Feb. 15, 2001

Related U.S. Application Data
(63) Continuation-in-part of application No. 09/431,099, filed on Nov. 1, 1999.

(30) Foreign Application Priority Data
Sep. 1, 1999 (DE) 199 41 478.5

(51) Int. Cl. C12P 13/04; C12N 9/10; C07H 21/04; C12N 1/21

(52) U.S. Cl. 435/106; 435/193; 435/252.1; 536/23.2

(57) ABSTRACT

The invention relates to preferably recombinant DNA derived from Corynebacterium and replicable in coryneform microorganisms, which contains at least one nucleotide sequence that codes for the thrE gene, and a process for the production of L-threonine, which is characterised in that the following steps are carried out:

a) Fermentation of microorganisms in which at least the thrE gene is amplified (overexpressed), optionally in combination with further genes,

b) Enrichment of the L-threonine in the medium or in the cells of the microorganisms, and

c) Isolation of the L-threonine.

The present invention relates to nucleotide sequences coding for the thrE gene and a process for the enzymatic production of L-threonine using coryneform bacteria, in which the thrE gene is amplified.

### PRIOR ART

L-threonine is used in animal nutrition, in human medicine and in the pharmaceutical industry.

It is known that L-threonine can be produced by fermentation of strains of coryneform bacteria, in particular Corynebacterium glutamicum. On account of the great importance of L-threonine, attempts are constantly being made to improve the production processes. Production improvements may relate to fermentation technology measures such as for example stirring and provision of oxygen, or the composition of the nutrient medium such as for example the sugar concentration during fermentation, or the working-up to the product form by for example ion exchange chromatography, or the intrinsic production properties of the microorganism itself.

Methods employing mutagenesis, selection and choice of mutants are used to improve the production properties of these microorganisms. In this way strains are obtained that are resistant to antimetabolites such as for example the threonine analogon α-amino-β-hydroxyvaleric acid (AHV)or are auxotrophic for regulatory significant amino acids and produce L-threonine.

For some years now recombinant DNA technology methods have also been used for the strain improvement of L-threonine producing strains of Corynebacterium, by amplifying individual threonine biosynthesis genes and investigating the action on L-threonine production.

### OBJECT OF THE INVENTION

The inventors have aimed to provide new measures for the improved enzymatic production of L-threonine.

### DESCRIPTION OF THE INVENTION

L-threonine is used in animal nutrition, in human medicine and in the pharmaceutical industry. There is therefore a general interest in providing new improved processes for producing L-threonine.

The object of the invention is a preferably recombinant DNA derived from Corynebacterium and replicable in coryneform microorganisms, which contains at least the nucleotide sequence coding for the thrE gene, represented in the sequences SEQ-ID-No. 1 and SEQ-ID-No. 3.

The object of the invention is also a replicable DNA according to claim 1 with:

(i) the nucleotide sequences shown in SEQ-ID-No. 1 or SEQ-ID-No. 3, that code for the thrE gene, or

(ii) at least one sequence that corresponds to the sequences (i) within the degeneration region of the genetic code, or

(iii) at least one sequence that hybridises with the sequence complementary to the sequences (i) or (ii), and/or optionally

(iv) functionally neutral sense mutations in (i).

The object of the invention are also coryneform microorganisms, in particular of the genus Corynebacterium, transformed by the introduction of the aforementioned replicable DNA.

The invention finally relates to a process for the enzymatic production of L-threonine using coryneform bacteria, which in particular already produce L-threonine and in which the nucleotide sequence(s) coding for the thrE gene is/are amplified, in particular overexpressed.

The term “amplification” describes in this connection the enhancement of the intracellular activity of one or more enzymes in a microorganism that are coded by the corresponding DNA, by for example increasing the copy number of the gene or genes or using a strong promoter or a gene that codes for a corresponding enzyme having a high activity, and if necessary using a combination of these measures.

The microorganisms that are the object of the present invention can produce L-threonine from glucose, sucrose, lactose, fructose, maltose, molasses, starch, cellulose or from glycerol and ethanol. The microorganisms may be representatives of coryneform bacteria, in particular of the genus Corynebacterium. In the genus Corynebacterium the species Corynebacterium glutamicum should in particular be mentioned, which is known to those in the specialist field for its ability to produce L-amino acids.

Suitable strains of the genus Corynebacterium, in particular of the species Corynebacterium glutamicum, are in particular the known wild type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020 and L-threonine-producing mutants or strains obtained thereform, for example

Corynebacterium glutamicum ATCC21649

Brevibacterium flavum BB69

Brevibacterium flavum DSM5399

Brevibacterium lactofermentum FERM-BP 269

Brevibacterium lactofermentum TBB-10

The inventors have successfully managed to isolate the thrE gene of Corynebacterium glutamicum. In order to isolate the thrE gene a mutant of C. glutamicum defective in the thrE gene is first of all produced. To this end a suitable starting strain such as for example ATCC14752 or ATCC13032 is subjected to a mutagenesis process.

Conventional mutagenesis processes include treatment with chemicals, for example N-methyl-N-nitro-N-nitrosoguanidine, or UV irradiation. Such processes for initiating mutation are generally known and may be consulted in, inter alia, Miller (A Short Course in Bacterial Genetics, A Laboratory Manual and Handbook for Escherichia coli and Related Bacteria (Cold Spring Harbor Laboratory Press, 1992)) or in the handbook “Manual of Methods for General Bacteriology” The American Society for Bacteriology (Washington D.C., USA, 1981).

Another mutagenesis process is the method of transposon mutagenesis in which the property of a transposon is utilised to “jump” in DNA sequences and thereby interfere with or switch off the function of the relevant gene. Transposons of coryneform bacteria are known in the specialist field. For example, the erythromycin resistance transposon Tn5432 (Tauch et al., Plasmid (1995) 33:168-179) and the chloramphenicol resistance transposon Tn5546 have been isolated from Corynebacterium xerosis strain M82B.

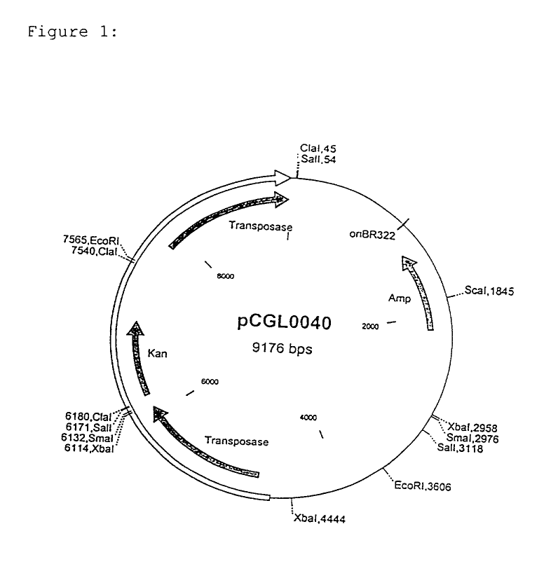

Another transposon is the transposon Tn5531 described by Ankri et al. (Journal of Bacteriology (1996) 178:4412-4419) and that was used for example in the course of the present invention. The transposon Tn5531 contains the aph3 kanamycin resistance gene and can be delivered for example in the form of the plasmid vector pCGL0040, which is shown in FIG. 1. The nucleotide sequence of the transposon Tn5531 is freely available under the accession number U53587 from the National Center for Biotechnology Information (NCBI, Bethesda, Md., USA).

After mutagenesis, preferably transposon mutagenesis, has been carried out a search is made for a mutant defective in the thrE gene. A mutant defectve in the thrE gene is recognised by the fact that it exhibits good growth on minimal agar, but poor growth on minimal agar that has been supplemented with threonine-containing oligopeptides, for example the tripeptide threonyl-threonyl-threonine.

An example of such a mutant is the strain ATCC14752ΔilvAthrE::Tn5531.

A strain produced in the described manner may be used to isolate and clone the thrE gene.

To this end a gene bank of the bacterium that is of interest may be established. The establishment of gene banks is recorded in generally known textbooks and manuals. There may be mentioned by way of example the textbook by Winnacker: Gene und Klone, eine Einfuhrung in die Gentechnologie (Gene and Clones, An Introduction to Gene Technology) (Verlag Chemie, Weinheim, Germany, 1990) or the manual by Sambrook et al.: Molecular Cloning, A Laboratory Manual (Cold Spring Harbor Laboratory Press, 1989). A very well-known gene bank is that of the E. coli K-12 strain W3110, which has been established by Kohara et al. (Cell 50, 495-508 (1987)) in λ-vectors. Bathe et al. (Molecular and General Genetics, 252:255-265, 1996) describes a gene bank of C. glutamicum ATCC13032, which has been established in the E. coli K-12 strain NM554 (Raleigh et al., 1988, Nucleic Acids Research 16:1563-1575) with the aid of the cosmid vector SuperCos I (Wahl et al., 1987, Proceedings of the National Academy of Sciences USA, 84:2160-2164). For the present invention those vectors are suitable that replicate in coryneform bacteria, preferably Corynebacterium glutamicum. Such vectors are known from the prior art; the plasmid vector pZ1 may be mentioned as an example, which is described by Menkel et al. (Applied and Environmental Microbiology (1989) 64:549-554). The gene bank obtained in the described way is then converted by means of transformation or electroporation into the indicator strain defective in the thrE gene and those transformants are sought that have the ability to grow on minimal agar in the presence of threonine-containing oligopeptides. The cloned DNA fragment may then be subjected to a sequence analysis.

When using a mutant of a coryneform bacterium produced by Tn5531 mutagenesis, for example the strain ATCC14752ΔilvAthrE::Tn553, the thrE::Tn5531 allele may be cloned directly using the kanamycin resistance gene aph3 contained in the latter and isolated. For this purpose known cloning vectors are used, such as for example pUC18 (Norrander et al., Gene (1983) 26:101-106 and Yanisch-Perron et al., Gene (1985) 33:103-119). Particularly suitable as cloning hosts are those E. coli strains that are both restriction-defective and recombinant-defective. An example is the strain DH5αmcr, which has been described by Grant et al. (Proceedings of the National Academy of Sciences USA, 87 (1990) 4645-4649). The selection for transformants is carried out in the presence of kanamycin. The plasmid DNA of the resultant transformants is then sequenced. For this purpose the dideoxy chain termination method described by Sanger et al. may be used (Proceedings of the National Academy of Sciences of the United States of America USA (1977) 74:5463-5467). The thrE gene sequences upstream and downstream of the Tn5531 insertion site are thereby obtained. The resultant nucleotide sequences are then analysed and assembled with commercially available sequence analysis programs, for example with the program package Lasergene (Biocomputing Software for Windows, DNASTAR, Madison, USA) or the program package HUSAR (Release 4.0, EMBL, Heidelberg, Germany).

In this way the new DNA sequence of C. glutamicum coding for the thrE gene was obtained, which as SEQ ID NO 1 is a constituent part of the present invention. The amino acid sequence of the corresponding protein has also been derived from the present DNA sequence using the aforedescribed methods. The resulting amino acid sequence of the thrE gene product is represented in SEQ ID NO 2.

Coding DNA sequences that are produced from SEQ ID NO 1 by the degenerability of the genetic code are likewise a constituent part of the invention. In the same way, DNA sequences that hybridise with SEQ ID NO 1 or parts of SEQ ID NO 1 are a constituent part of the invention. Furthermore, conservative amino acid exchanges, for example the exchange of glycine by alanine or of aspartic acid by glutamic acid in proteins are known in the specialist field as sense mutations, which do not cause any fundamental change in the activity of the protein, i.e. are functionally neutral. It is furthermore known that changes at the N- and/or the C-terminus of a protein do not substantially affect its function or may even stabilise it. The person skilled in the art may find details of this in, inter alia, Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks on genetics and molecular biology. Amino acid sequences that are produced in a corresponding manner from SEQ ID NO 2 are likewise a constituent part of the invention.

Suitable primers can be synthesised using the nucleotide sequence shown in SEQ ID NO. 1 and these are then used to amplify by means of the polymerase chain reaction (PCR) thrE genes of various coryneform bacteria and strains. The person skilled in the art may find details of this in for example the manual by Gait: Oligonucleotide synthesis: a practical approach (IRL Press, Oxford, UK, 1984) and in Newton and Graham: PCR (Spektrum Akademischer Verlag, Heidelberg, Germany, 1994). Alternatively, the nucleotide sequence shown in SEQ ID NO. 1 or parts thereof may be used as a probe to search for thrE genes in gene banks of in particular coryneform bacteria. The person skilled in the art can find details of this in for example the manual “The DIG System Users Guide for Filter Hybridization” published by Boehringer Mannheim GmbH (Mannheim, Germany, 1993) and in Liebl et al. (International Journal of Systematic Bacteriology (1991) 41:255-260). The thrE gene-containing DNA fragments amplified in this way are then cloned and sequenced.

The DNA sequence of the thrE gene of the strain ATCC13032 illustrated in SEQ ID NO. 3 was obtained in this way, and is likewise a constituent part of the present invention. The resultant amino acid sequence is shown in SEQ ID NO. 4.

The invention also provides a process for isolating the thrE gene, characterised in that mutants, preferably of coryneform bacteria, defective in the thrE gene are obtained as indicator strains that do not grow or grow only weakly on a nutrient medium containing a threonine-containing oligopeptide, and

a) the thrE gene is identified and isolated after establishing a gene bank, or

b) in the case of transposon mutagenesis is selected for the transposon preferably exhibiting resistance to antibiotics, and the thrE gene is thereby obtained.

The inventors discovered from this that coryneform bacteria after over-expression of the thrE gene produce L-threonine in an improved manner

In order to achieve an over-expression, the copy number of the corresponding genes can be increased, or the promoter and regulation region or the ribosome binding site located upstream of the structure gene can be mutated. Expression cassettes that are incorporated upstream of the structure gene work in the same way. It is in addition possible to enhance the expression during the course of the enzymatic L-threonine production by inducible promoters. The expression is also improved by measures aimed at lengthening the lifetime of the m-RNA. The enzymatic activity can also be increased by preventing the decomposition of the enzyme protein. The genes or gene constructs may be present either in plasmids with different copy numbers or may be integrated and amplified in the chromosome. Alternatively, an over-expression of the relevant genes can also be achieved by changing the composition of the culture media and cultivation conditions.

A person skilled in the art can find details of this in, inter alia, Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European Patent Specification EPS 0 472 869, in U.S. Pat. No. 4,601,893, in Schwarzer and Pühler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in patent application WO 96/15246, in Malumbres et al. (Gene 134, 15-24 (1993)), in Japanese laid-open specification JP-A-10-229891, in Jensen and Hammer (Biotechnology and Bioengineering 58, 191-195 (1998)), in Makrides (Microbiological Reviews 60:512-538 (1996)) and in known textbooks on genetics and molecular biology.

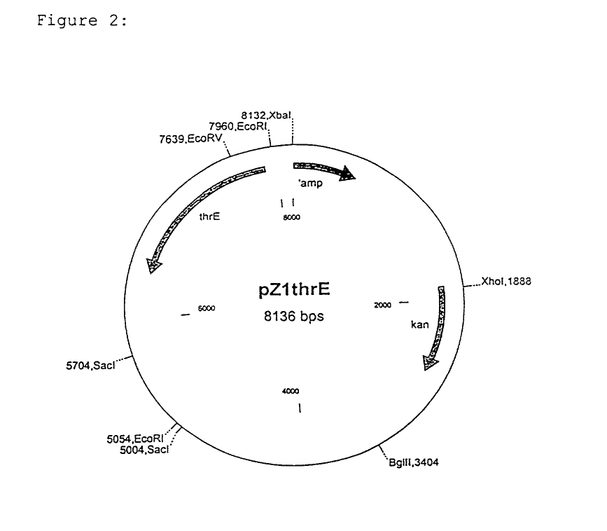

An example of a plasmid by means of which the thrE gene can be overexpressed is pZ1thrE (FIG. 2), which is contained in the strain DM368-2 pZ1thrE. Plasmid pZ1thrE is a C. glutamicum—E. coli shuttle vector based on plasmid pZ1, which is described by Menkel et al. (Applied and Environmental Microbiology (1989) 64:549-554). Other plasmid vectors replicable in C. glutamicum, such as for example pEKE×1 (Eikmanns et al., Gene 102:93-98 (1991)) or pZ8-1 (EP-B-0 375 889) can be used in the same way.

In addition, it may be advantageous for the production of L-threonine to over-express, in addition to the new thrE gene, one or more enzymes of the known threonine biosynthesis pathway or enzymes of the anaplerotic metabolism or enzymes of the citric acid cycle. The following may for example be simultaneously overexpressed:

the hom gene coding for homoserine dehydrogenase (Peoples et al., Molecular Microbiology 2, 63-72 (1988)) or the hom^{dr} allele coding for a feedback-resistant homoserine dehydrogenase (Archer et al.Gene 107, 53-59, (1991)), or

the pyc gene (DE-A-19 831 609) coding for pyruvate carboxylase, or

the mqo gene coding for malate:quinone oxidoreductase (Molenaar et al., European Journal of Biochemistry 254, 395-403 (1998)).

For the production of L-threonine it may furthermore be advantageous, in addition to the over-expression of the thrE gene, to exclude undesirable secondary reactions, such as for example the threonine-dehydrogenase reaction (Nakayama: “Breeding of Amino Acid Producing Micro-organisms”, in: Overproduction of Microbial Products, Krumphanzl, Sikyta, Vanek (eds.), Academic Press, London, UK, 1982 and Bell and Turner, Biochemical Journal 156, 449-458 (1976)).

The microorganisms produced according to the invention may be cultivated continuously or batchwise in a batch process (batch cultivation) or in a fed batch (feed process) or repeated fed batch process (repetitive feed process) for the purposes of producing L-threonine. A summary of known cultivation methods is given in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Brunswick/Wiesbaden, 1994)).

The culture medium to be used must satisfy in an appropriate manner the requirements of the respective strains. Descriptions of culture media for various microorganisms are given in “Manual of Methods for General Bacteriology” The American Society for Bacteriology (Washington D.C., USA, 1981). Sources of carbon that may be used include sugars and carbohydrates, for example glucose, sucrose, lactose, fructose, maltose, molasses, starch and cellulose, oils and fats such as soybean oil, sunflower oil, groundnut oil and coconut oil, fatty acids such as palmitic acid, stearic acid and linoleic acid, alcohols such as glycerol and ethanol, and organic acids such as acetic acid. These substances may be used individually or as a mixture. Sources of nitrogen that may be used include organic compounds containing nitrogen such as peptones, yeast extract, meat extract, malt extract, corn steep liquor, soybean meal and urea, or inorganic compounds such as ammonium sulfate, ammonium chloride, ammonium phosphate, ammonium carbonate and ammonium nitrate. The sources of nitrogen may be used individually or as a mixture. Sources of phosphorus that may be used include phosphoric acid, potassium dihydrogen phosphate or dipotassium hydrogen phosphate, or the corresponding sodium salts. The culture medium must furthermore contain salts of metals such as for example magnesium sulfate or iron sulfate, which are necessary for growth. Finally, essential growth substances such as amino acids and vitamins may be used in addition to the aforementioned substances. Moreover, suitable precursors may be added to the culture medium. The aforementioned substances may be added to the culture in the form of a single batch or in an appropriate manner during the cultivation.

Basic compounds such as sodium hydroxide, potassium hydroxide, ammonia or ammonia water, or acidic compounds such as phosphoric acid or sulfuric acid may be added in an appropriate manner in order to control the pH of the culture. Anti-foaming agents such as for example fatty acid polyglycol esters may be used to control foam formation. Suitable selectively acting substances, for example antibiotics, may be added to the medium in order to maintain the stability of plasmids. Oxygen or oxygen-containing gas mixtures, for example air, may be fed into the culture to maintain aerobic conditions. The temperature of the culture is normally 20° C. to 45° C., and preferably 25° C. to 40° C. Cultivation is continued until a maximum amount of L-threonine has been formed. This target is normally achieved within 10 to 160 hours.

The analysis of L-threonine can be carried out by anion exchange chromatography followed by ninhydrin derivatisation as described by Spackman et al. (Analytical Chemistry, 30, (1958), 1190), or can be carried out by reversed phase HPLC as described by Lindroth et al. (Analytical Chemistry (1979) 51:1167-1174).

The following microorganisms have been registered according to the Budapest Treaty at the German Collection for Microorganisms and Cell Cultures (DSMZ, Brunswick, Germany):

Brevibacterium flavum strain DM368-2 pZ1thrE as DSM 12840

Escherichia coli strain GM2929pCGL0040 as DSM 12839

### EXAMPLES

The present invention is described in more details hereinafter with the aid of examples of implementation.

The isolation of plasmid DNA from Escherichia coli as well as all techniques for restriction, Klenow and alkaline phosphatase treatment were carried out according to Sambrook et al. (Molecular cloning. A laboratory manual (1989) Cold Spring Harbour Laboratory Press). Unless otherwise specified, the transformation of Escherichia coli was carried out according to Chung et al. (Proceedings of the National Academy of Sciences of the United States of America USA (1989) 86:2172-2175).

### Example 1

Cloning and sequencing of the thrE gene of Corynebacterium glutamicum ATCC14752

1. Transposon Mutagenesis and Choice of Mutants

The strain Corynebacterium glutamicum ATCC14752ΔilvA was subjected to mutagenesis with the transposon Tn5531, whose sequence is filed under Accession No. U53587 in the Nucleotide Databank of the National Center for Biotechnology Information (Bethesda, USA). The incorporation of a deletion into the ilvA gene of Corynebacterium glutamicum ATCC14752 was carried out with the gene exchange system described by Schafer et al. (Gene (1994) 145:69-73). To this end, the inactivation vector pK19mobsacBΔilvA (Applied and Environmental Microbiology (1999) 65:1973-1979) constructed by Sahm et al. was used for the deletion. The methylase-defective Escherichia coli strain SCS110 (Jerpseth and Kretz, STRATEGIES in molecular biology 6, 22, (1993)) from Stratagene (Heidelberg, Germany) was first of all transformed with 200 ng of the vector pK19mobsacBΔilvA. Transformants were identified by means of their kanamycin resistence on 50 μg/mL kanamycin-containing LB-agar plates. The plasmid pK19mobsacBΔilvA was prepared from one of the transformants. This inactivation plasmid was then introduced into the strain Corynebacterium glutamicum ATCC14752 by means of electroporation (Haynes et al., FEMS Microbiology Letters (1989) 61:329-334). Clones in which the inactivation vector was present integrated in the genome were identified by means of their kanamycin resistance on 15 μg/mL kanamycin-containing LBHIS-agar plates (Liebl et al., FEMS Microbiology Letters (1989) 65:299-304). In order to select for the excision of the vector, kanamycin-resistant clones were plated out on sucrose-containing LBG-Medium (LB-Medium with 15 g/L agar , 2% glucose and 10% sucrose). Colonies were obtained in this way which had lost the vector through a second recombination event (Jäger et al.; Journal of Bacteriology (1992) 174:5462-5465). By hetero-inoculation on minimal medium plates (CGXII-Medium with 15 g/L agar (Keilhauer et al., Journal of Bacteriology (1993) 175:5595-5603)) with and without 300 mg/L of L-isoleucine, and with and without 50 μg/mL of kanamycin, six clones were isolated that by excision of the vector were kanamycin sensitive and isoleucine auxotrophic and in which only the incomplete ilvA-Gen (ΔilvA allele) was present in the genome. One of these clones was designated strain ATCC14752ΔilvA and used for the transposon mutagenesis.

The plasmid pCGL0040, which contains the assembled transposon Tn5531 (Ankri et al., Journal of Bacteriology (1996) 178:4412-4419) was isolated from the methylase-defective E. coli strai GM2929pCGL0040 (E. coli GM2929: Palmer et al., Gene (1994) 143:1-12). The strain Corynebacterium glutamicum ATCC14752ΔilvA was transformed with the plasmid pCGL0040 by means of electroporation (Haynes et al., FEMS Microbiology Letters (1989) 61:329-334). Clones in which the transposon Tn5531 was integrated into the genome were identified by means of their kanamycin resistance on 15 μg/mL kanamycin-containing LBHIS-agar plates (Liebl et al., FEMS Microbiology Letters (1989) 65:299-304). In this way 2000 clones were obtained which were checked for retarded growth in the presence of threonyl-threonyl-threonine. For this purpose all clones were transferred individually to CGXII minimal medium agar plates with and without 2 mM threonyl-threonyl-threonine. The medium was identical to the medium CGXII described by 5 Keilhauer et al. (Journal of Bacteriology (1993) 175:5593-5603), but in addition contained 25 μg/mL of kanamycin, 300 mg/L of L-isoleucine and 15 g/L of agar. The composition of the medium described by Keilhauer et al. is shown in Table 1.

TABLE 1
Composition of the Medium CCXII

| Component | Concentration | |
|---|---|---|
| (NH₄)₂SO₄ | 20 | g/L |
| Urea | 5 | g/L |
| KH₂PO₄ | 1 | g/L |
| K₂HPO₄ | 1 | g/L |
| MgSO₄ × 7 H₂O | 0.25 | g/L |
| 3-morpholinopropanesulfonic acid | 42 | g/L |
| CaCl₂ | 10 | mg/L |
| FeSO₄ × 7 H₂O | 10 | mg/L |
| MnSO₄ × H₂O | 10 | mg/L |
| ZnSO₄ × 7H₂O | 1 | mg/L |
| CuSO₄ | 0.2 | mg/L |
| NiCl₂ × 6 H₂O | 0.02 | mg/L |
| Biotin | 0.2 | mg/L |
| Glucose | 40 | g/L |
| Protocatechuic Acid | 30 | mg/L |

The agar plates were incubated at 30° C. and the growth was investigated after 12, 18 and 24 hours. A transposon mutant was obtained that grew in a comparable manner to the initial strain Corynebacterium glutamicum ATCC14752ΔilvA without threonyl-threonyl-threonine, but which in the presence of 2 mM threonyl-threonyl-threonine exhibited retarded growth. This was designated ATCC14752ΔilvAthrE::Tn5531.

2. Cloning and sequencing of the insertion site of Tn5531 in ATCC14752ΔilvAthrE::Tn5531.

In order to clone the insertion site located upstream of the transposon Tn5531 in the mutant described in Example 1.1, the chromosomal DNA of this mutant strain was first of all isolated as described by Schwarzer et al. (Bio/Technology (1990) 9:84-87) and 400 ng of the latter was cut with the restriction endonuclease EcoRI. The complete restriction insert was ligated with the vector pUC18 likewise linearised with EcoRI (Norander et al., Gene (1983) 26:101-106) from Roche Diagnostics (Mannheim, Germany). The E. coli strain DH5αmcr (Grant et al., Proceedings of the National Academy of Sciences of the United States of America USA (1990) 87:4645-4649) was transformed with the complete ligation insert by means of electroporation (Dower et al., Nucleic Acid Research (1988) 16:6127-6145). Transformants in which the insertion sites of the transposon Tn5531 were present cloned on the vector pUC18 were identified by means of their carbenicillin resistance and kanamycin resistance on LB-agar plates containing 50 μg/mL of carbenicillin and 25 μg/mL of kanamycin. The plasmids were prepared from three of the transformants and the sizes of the cloned inserts were determined by restriction analysis. The nucleotide sequence of the insertion site on one of the plasmids was determined with a ca. 5.7 kb large insert by the dideoxy chain termination method of Sanger et al. (Proceedings of the National Academy of Sciences of the United States of America USA (1977) 74:5463-5467). For this purpose 2.2 kb of the insert were sequenced starting from the following oligonucleotide primer: 5′-CGG GTC TAC ACC GCT AGC CCA GG-3′.

In order to identify the insertion site located downstream of the transposon, the chromosomal DNA of the mutant was cut with the restriction endonuclease XbaI and ligated in the vector pUC18 linearised with XbaI. The further cloning was carried out as described above. The nucleotide sequence of the insertion site on one of the plasmids was determined with a ca. 8.5 kb large insert by the dideoxy chain termination method of Sanger et al. (Proceedings of the National Academy of Sciences of the United States of America USA (1977) 74:5463-5467). For this purpose 0.65 kb of the insert was sequenced starting from the following oligonucleotide primer: 5′-CGG TGC CTT ATC CAT TCA GG-3′.

The obtained nucleotide sequences were analysed and assembled with the program package Lasergene (Biocomputing Software for Windows, DNASTAR, Madison, USA). This nucleotide sequence is reproduced as SEQ ID NO 1. The analysis identified an open reading frame 1467 bp long. The corresponding gene was designated the thrE gene. The associated gene product comprises 489 amino acids and is reproduced as SEQ ID NO 2.

### Example 2

Cloning and Sequencing of the Gene thrE from Corynebacterium glutamicum ATCC13032

The gene thrE was cloned in the E. coli cloning vector pUC18 (Norrander et al., Gene (1983) 26:101-106, Roche Diagnostics, Mannheim, Germany). The cloning was carried out in two stages. The gene from Corynebacterium glutamicum ATCC13032 was first of all amplified by a polymerase chain reaction (PCR) by means of the following oligonucleotide primer derived from SEQ ID NO 1 ThrE-forward:

5′-CCC CTT TGA CCT GGT GTT ATT G-3′ thrE-reverse:

5′-CGG CTG CGG TTT CCT CTT-3′

The PCR reaction was carried out in 30 cycles in the presence of 200 μM of deoxynucleotide triphosphates (dATP, dCTP, dGTP, dTTP) and, for each, 1 μM of the corresponding oligonucleotide, 100 ng of chromosomal DNA from Corynebacterium glutamicum ATCC13032, 1/10 volumes of 10-fold reaction buffer and 2.6 units of a heat-stable Taq-/Pwo-DNA polymerase mixture (Expand High Fidelity PCR System from Roche Diagnostics, Mannheim, Germany) in a Thermocycler (PTC-100, M J Research, Inc., Watertown, USA) under the following conditions: 94° C. for 30 seconds, 58° C. for 30 seconds and 72° C. for 2 minutes.

The amplified, about 1.9 kb large fragment was then ligated using the SureClone Ligation Kit (Amersham Pharmacia Biotech, Uppsala, Sweden) according to the manufacturer's instructions, into the SmaI cleavage site of the vector pUC18. The E. coli strain DH5αmcr (Grant et al., Proceedings of the National Academy of Sciences of the United States of America USA (1990) 87:4645-4649) was transformed with the whole ligation insert. Transformants were identified on the basis of their carbenicillin resistance on 50 μg/mL carbenicillin-containing LB agar plates. The plasmids were prepared from 8 of the tranformants and tested by restriction analysis for the presence of the 1.9 kb PCR fragment as insert. The resultant recombinant plasmid is designated hereinafter as pUC18thrE.

The nucleotide sequence of the 1.9 kb PCR fragment in plasmid pUC18thrE was determined by the dideoxy chain termination method of Sanger et al. (Proceedings of the National Academy of Sciences of the United States of America USA (1977) 74:5463-5467). For this purpose the complete insert of pUC18thrE was sequenced with the aid of the following primers from Roche Diagnostics (Mannheim, Germany).

Universal primer:

5′-GTA AAA CGA CGG CCA GT-3′

Reverse primer:

5′-GGA AAC AGC TAT GAC CAT G-3′

The nucleotide sequence is reproduced as SEQ ID NO 3. The contained nucleotide sequence was analysed using the program package Lasergene (Biocomputing Software for Windows, DNASTAR, Madison, USA). The analysis identified an open reading frame 1467 bp long, which was designated the thrE gene. This codes for a polypeptide of 489 amino acids, which is reproduced as SEQ ID NO 4.

### Example 3

Expression of the Gene thrE in Corynebacterium glutamicum

The gene thrE from Corynebacterium glutamicum ATCC13032 described in Example 2 was cloned for expression in the vector pZ1 (Menkel et al., Applied and Environmental Microbiology (1989) 64:549-554). For this purpose a 1881 bp large DNA fragment containing the gene thrE was excised from the plasmid pUC18thrE using the restriction enzymes SacI and XbaI. The 5′- and 3′-ends of this fragment were treated with Klenow enzyme. The resulting DNA fragment was ligated in the vector pZ1 previously linearised and dephosphorylated with ScaI. The E. coli strain DH5αamcr (Grant et al., Proceedings of the National Academy of Sciences of the United States of America USA (1990) 87:4645-4649) was transformed with the whole ligation insert. Tranformants were identified on the basis of their kanamycin resistance on 50 μg/mL kanamycin-containing LB agar plates. The plasmids were prepared from two transformants and checked by restriction analysis for the presence of the 1881 bp ScaI/XbaI fragment as insert. The recombinant plasmid produced in this way was designated pZ1thrE (FIG. 2).

The plasmids pZ1 and pZ1thrE were incorporated by means of electroporation (Haynes et al., FEMS Microbiology Letters (1989) 61:329-334) into the threonine-forming strain Brevibacterium flavum DM368-2. The strain DM368-2 is described in EP-B-0 385 940 and is filed as DSM5399. Transformants were identified on the basis of their kanamycin resistance on 15 μg/mL kanamycin-containing LBHIS-agar plates (Liebl et al., FEMS Microbiology Letters (1989) 65:299-304). The strains Brevibacterium flavum DM368-2 pZ1 and DM368-2 pZ1thrE were obtained in this way.

### Example 5

Preparation of L-threonine with Brevibacterium flavum

In order to investigate their threonine formation the strains B. flavum DM368-2 pZ1 and DM368-2 pZ1thrE were precultivated in 100 mL of brain heart infusion medium together with 50 μg of kanamycin/mL (Difco Laboratories, Detroit, USA) for 14 hours at 30° C. The cells were then washed once with 0.9% (w/v) of sodium chloride solution and 60 mL portions of CgXII medium were inoculated with this suspension so that the OD₆₀₀ (optical density at 600 nm) was 0.5. The medium was identical to the medium described by Keilhauer et al. (Journal of Bacteriology (1993) 175:5593-5603), but contained in addition 50 μg of kanamycin per mL. Both strains were cultivated at 30° C. over a period of 72 hours. Samples were taken after 0, 24, 48 and 72 hours and the cells were quickly centrifuged off (5 minutes at 13000 RPM in a Biofuge pico from Heraeus, Osterode, Germany).

The quantitative determination of the extracellular amino acid concentrations from the culture supernatant was carried out by means of reversed phase HPLC (Lindroth et al., Analytical chemistry (1979) 51:1167-1174). An HPLC apparatus of the HP1100 Series (Hewlett-Packard, Waldbronn, Germany) with attached fluorescence detector (G1321A) was used; the operation of the systems and the evaluation of the data was carried out with a HP-Chem-Station (Hewlett-Packard). 1 μL of the amino acid solution to be analysed was mixed in an automatic preliminary column derivatisation step with 20 μL of o-phthalaldehyde/2-mercaptoethanol reagent (Pierce Europe BV, Oud-Beijerland, Netherlands).

The resultant fluorescing, thio-substituted isoindoles (Jones et al., Journal of Chromatography (1983) 266:471-482) were separated in a combined preliminary column (40×4 mm Hypersil ODS 5) and main column (Hypersil ODS 5, both columns obtained from CS-Chromatographie Service GmbH, Langerwehe, Germany) using a gradient program with an increasingly non-polar phase (methanol). The polar eluent was sodium acetate (0.1 molar, pH 7,2); the flow rate was 0.8 mL per minute. The fluorescence detection of the derivatised amino acids was carried out at an excitation wavelength of 230 nm and an emission wavelength of 450 nm. The amino acid concentrations were calculated by comparison with an external standard and asparagine as additional internal standard.

The results are shown in Table 2.

TABLE 2

L-Threonine (g/L)

| Strain | 0 Hrs. | 24 Hrs. | 48 Hrs. | 72 Hrs. |
|---|---|---|---|---|
| DM368-2pZl | 0 | 0.46 | 1.27 | 1.50 |
| DM368-2 pZlthrE | 0 | 0.68 | 1.71 | 2.04 |

### Example 6

### Preparation of the vector pEC-t18mob2thrE

1. ° Construction of pEC-T18mob2

The E. coli glutamicum shuttle vector pEC-T18mob2 was constructed according to the prior art. The vector contains the replication region rep of the plasmid pGA1 including the replication effector (per U.S. Pat. No. 5,175,108; Nesvera et al., Journal of Bacteriology 179, 1525-15322 (1997)), the tetracycline resistance-imparting tetA(Z)gene of the plasmid pAG1 (U.S. Pat. No. 5,158,891; gene bank registration at the National Center for Biotechnology Information (NCBI, Bethesda, Md., USA) with the accession number AF121000), the replication region oriV of the plasmid pMB1 (Sutcliffe, Cold Spring Harbor Symposium on Quantitative Biology 43, 77-90 (1979)), the lacZα gene fragment including the lac promoter and a multiple cloning site, mcs (Norrander et al. Gene 26, 101-106 (1983)) and the mob region of the plasmid RP4 (Simon et al., (1983) Bio/Technology 1:784-791).

The constructed vector was transformed into the E. coli strain DH5α (Hanahan, In: DNA cloning. A practical approach. Vol. 1. IRL-Press, Oxford, Washington DC, USA, 1985). The selection of plasmid-carrying cells was effected by plating out the transformation batch onto LB agar (Sambrook et al., Molecular cloning: a laboratory manual. 2^{nd} Ed. Cold Spring Harbor Laboratory Press, Cold Spring Harbor, N.Y., USA, 1989) that had been supplemented with 5 mg/1 tetracycline. Plasmid DNA was isolated from a transformant by means of a QIAprep Spin Miniprep Kit from Qiagen and was checked by restriction with the restriction enzyme EcoRI and HindIII followed by agarose gel electrophoresis (0.8%).

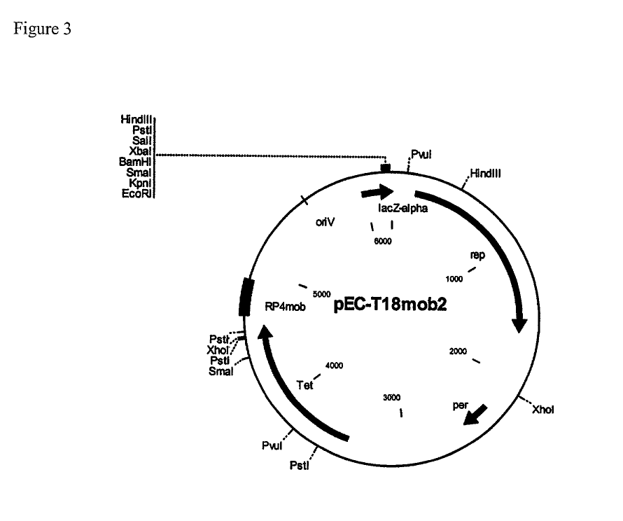

The plasmid was named pEC-T18mob2 and is illustrated in FIG. 3.

2. Construction of pEC-T18mob2thrE

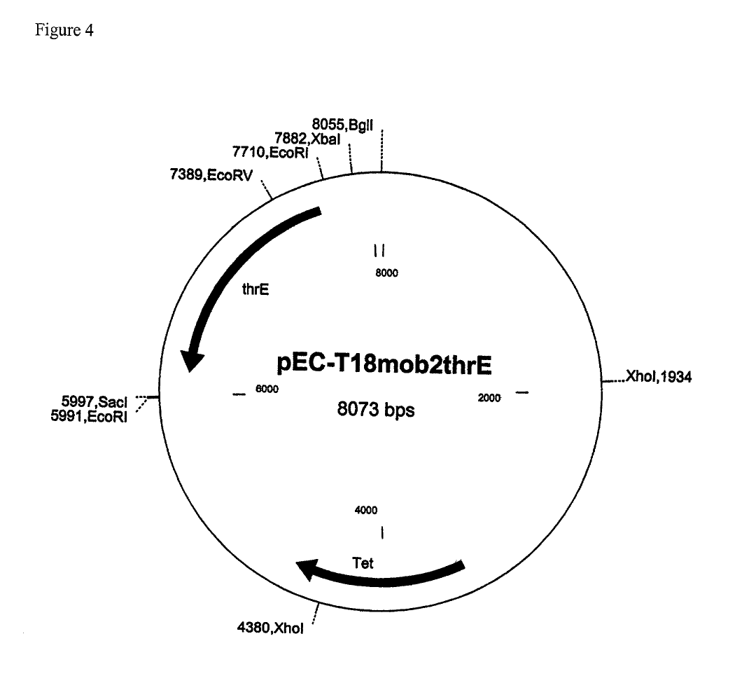

The gene thrE from Corynebacterium glutamicum ATCC13032 described in Example 2 was cloned for expression into the vector pEC-T18mob2. For this purpose an 1881 bp large DNA fragment containing the gene thrE was cut out from the plasmid pUC18thrE using the restriction enzymes SacI and XbaI. The resulting DNA fragment was ligated into the vector pEC-T18mob2 that had previously been linearised and dephosphorylated with SacI and XbaI. The E. coli strain DHSαmcr (Grant et al., Proceedings of the National Academy of Sciences of the United States of America USA (1990) 87:4645-4649) was transformed with the whole ligation batch. Transformants were identified on the basis of their tetracycline resistance on LB agar plates containing 5 μg/mL tetracycline. The plasmids were prepared from two transformants and checked by restriction analysis for the presence of the 1881 bp ScaI/XbaI fragment as insert. The recombinant plasmid obtained in this way was designated pEC-T18mob2thrE (FIG. 4).

The plasmids pEC-T18mob2 and pEC-T18mob2thrE were introduced by means of electroporation (Haynes et al., FEMS Microbiology Letters (1989) 61:329-334) into the threonine-forming strain Corynebacterium glutamicum MH2022B-DR17 (Reinscheid et al., Applied and Environmental Microbiology (1994) 60:126-132). Tranformants were identified on the basis of their tetracycline resistance and kanamycin resistance on LBHIS agar plates containing 15 μg/mL kanamycin and 5 μg/mL tetracycline (Liebl et al., FEMS Microbiology Letters (1989) 65:299-304). The strains Corynebacterium-glutamicum MH20-22B-DR17/pEC-T18mob2 and MH20-22B-DR17/pEC-T18mob2thrE were obtained in this way.

### Example 7

### Preparation of L-threonine with Corynebacterium glutamicum

In order to investigate their threonine formation, the strains C. glutamicum MH20-22B-DR17/pEC-T18mob2 and MH2022B-DR17/pEC-T18mob2thrE were precultured for 14 hours at 30° C. in 100 mL brain heart infusion medium containing 25 μg kanamycin/mL and 5 μg tetracycline/mL (Difco Laboratories, Detroit, USA). The cells were then washed once with 0.9% (w/v) sodium chloride solution and 60 mL portions of CgXII medium were inoculated with this suspension so that the OD₆₀₀ (optical density at 600 nm) was 0.5. The medium was identical to that described by Keilhauer et al. (Journal of Bacteriology (1993) 175:5593-5603) but additionally contained 25 μg kanamycin and 5 μg tetracycline per mL. Both strains were cultured at 30° C. over a period of 72 hours. Samples were taken after 0, 24, 48 and 72 hours and the cells were briefly centrifuged off (5 minutes at 13000 revs per minute in a Biofuge pico centrifuge from Heraeus, Osterode, Germany).

The quantitative determination of the extracellular amino acid concentrations from the culture supernatant was carried out as already described in Example 5 for Brevibacterium flavum using reversed phase HPLC (Lindroth et al., Analytical chemistry (1979) 51:1167-1174). The amino acid concentrations were calculated by comparison with an external standard and asparagine as additional internal standard.

The results are given in Table 3.

TABLE 3

| | L-Threonine (g/L) | | | |
|---|---|---|---|---|
| Strain | 0 hrs. | 24 hrs. | 48 hrs. | 72 hrs. |
| MH20-22B-DR17/ | 0 | 3.42 | 5.82 | 5.78 |
| pEC-T18mob2 | | | | |
| MH20-22B-DR17/ | 0 | 4.16 | 7.53 | 8.05 |
| pEC-T18mob2thrE | | | | |

The following figures are included:

FIG. 1: Map of the plasmid pCGL0040 containing the transposon Tn553 1.

The transposon is characterised as a non-hatched arrow.

FIG. 2: Map of the plasmid pZ1thrE containing the thrE gene.

FIG. 3: Map of the plasmid pEC-T18mob2.

FIG. 4: Map of the plasmid pEC-T18mob2thrE containing the thrE gene.

Length data should be regarded as approximate. The abbreviations and references employed have the following meaning:

amp: ampicillin resistance gene

kan: kanamycin resistance gene

′amp: 3′ part of the ampicillin resistance gene

oriBR322: replication region of the plasmid pBR322

tet: resistance gene for tetracycline

oriV: plasmid-coded replication source of E. coli

PR4mob: mob region for mobilisation of the plasmid

rep: plasmid-coded replication source from C. glutamicum plasmid pGA1

per: gene for controlling the number of copies from pGA1

lacZ-alpha: lacZα gene fragment(N-terminus) of the β-galactosidase gene

The abbreviations for the restriction enzymes have the following meaning

BamHI: restriction endonuclease from Bacillus amyloliquefaciens

BglII: restriction endonuclease from Bacillus globigii

EcoRI: restriction endonuclease from Escherichia coli

EcoRV: restriction endonuclease from Escherichia coli

HindIII: restriction endonuclease from Haemophilus influenzae

KpnI: restriction endonuclease from Klebsiella pneumoniae

PstI: restriction endonuclease from Providencia stuartii

PvuI: restriction endonuclease from Proteus vulgaris

SacI: restriction endonuclease from Streptomyces achromogenes

SalI: restriction endonuclease from Streptomyces albus

SmaI: restriction endonuclease from Serratia marcescens

XbaI: restriction endonuclease from Xanthomonas badrii

XhoI: restriction endonuclease from Xanthomonas holcicola

1. Preferably recombinant DNA derived from Corynebacterium and replicable in coryneform microorganisms, which contains at least one nucleotide sequence that codes for the thrE gene.

2. Replicable DNA according to claim 1 with
(i) the mucleotide sequences shown in SEQ-ID-No. 1, or SEQ-ID No. 3, which code for the thrE gene, or
(ii) at least once sequence that corresponds to the sequences (i) within the degeneration region of the genetic code, or
(iii) at least once sequence that hybridises with the sequences complementary to the sequences (i) or (ii), and/or optionally
(iv) functionally neutral sense mutations in (i).

3. Amino acid sequence of the protein, derived from the nucleotide sequences according to claim 1 or 2, shown in SEQ-ID-No. 2 and in SEQ-ID-No. 4.

4. Coryneforme microorganisms, in particular of the genus Corynebacterium, transformed by the introduction of one or more of the replicable DNA according to claim 1 or 2.

5. Corynebacterium glutamicum DM368-2 pZ1thrE, filed under Number DSM 12840.

6. Process for producing L-threonine by fermentation of coryneform bacteria, characterised in that bacteria are used in which nucleotide sequences coding for the thrE gene are amplified, and in particular are overexpressed.

7. Process according to claim 6, characterised in that bacteria are used in which in addition one or more genes of the threonine biosynthesis pathway is/are amplified.

8. Process according to claims 6 and 7, characterised in that a strain transformed with a plasmid vector is used and the plasmid vector carries the nucleotide sequence coding for the thrE gene.

9. Process according to claims 6 and 8, characterised in that the thrE gene is overexpressed in microorganisms that contain further metabolite or antimetabolite resistance mutations.

10. Process according to claims 6 to 9, characterised in that the microorganisms in order to achieve over-expression are fermented in altered culture media, and/or the fermentation conditions are changed.

11. Process according to claims 6 to 10, characterised in that microorganisms are used in which the metabolic pathways that reduce threonine formation are at least partially switched off.

12. Process according to claims 6 to 11, characterised in that microorganisms are used in which in addition to the thrE gene the remaining genes of the metabolic pathway for threonine formation are amplified individually or jointly (overexpressed).

13. Process for producing L-threonine, characterised in that the following steps are carried out:
a) Fermentation of microorganisms according to one or more of the preceding claims, in which at least the thrE gene is amplified (overexpressed) optionally in combination with further genes,
b) Enrichment of the L-threonine in the medium or in the cells of the microorganisms, and
c) Isolation of the L-threonine.

14. Process according to one or more of the preceding claims, characterised in that microorganisms of the genus Corynebacterium are used.

15. Process for isolating the thrE gene, characterised in that mutants, preferably of coryneform bacteria, defective in the thrE gene that do not grow or grow only weakly on a nutrient medium containing a threonine-containing oligopeptide are obtained as indicator strains, and
a) the thrE gene is identified and isolated after establishing a gene bank, or
b) in the case of transposon mutagenesis is selected for the transposon preferably exhibiting resistance to antibiotics, and the thrE gene is thereby obtained.
